(19) United States
(12) Patent Application Publication
(10) Pub. No.: US 2025/0090707 A1
(43) Pub. Date: Mar. 20, 2025

(54) ROBOTIC AND AI-AUGMENTED IN SITU DETECTION AND DISINFECTION PLATFORM

(71) Applicant: University of South Florida, Tampa, FL (US)

(72) Inventors: Ying Zhong, Tampa, FL (US); Subhra Mohapatra, Tampa, FL (US); Shyam Mohapatra, Tampa, FL (US); Ehsan Sheybani, Tampa, FL (US); Tansel Yucelen, Tampa, FL (US)

(21) Appl. No.: 18/967,136

(22) Filed: Dec. 3, 2024

Related U.S. Application Data
(63) Continuation of application No. PCT/US2023/025422, filed on Jun. 15, 2023.
(60) Provisional application No. 63/366,445, filed on Jun. 15, 2022.

(51) Int. Cl. A61L 2/24; A61L 2/03

(52) U.S. Cl. CPC A61L 2/24; A61L 2/03; A61L 2202/14; A61L 2202/16

(57) ABSTRACT

An autonomous pathogen detection and disinfection mobile platform having a small footprint, such that embodiments of the platform are deployable within interior spaces such as hospital, clinics, and other healthcare facilities. The platform includes a detection chamber that efficiently identifies a presence of a pathogen, such as a microorganism. The platform also includes a disinfection discharge component, such as a discharge needle, that treats a surface that includes a presence of a pathogen. As an autonomous mobile device, the platform includes sensors, cameras, or similar devices to detect surrounding surfaces, such that a computing device disposed on the platform plots a dynamic path for the platform based on received feedback.

### CROSS-REFERENCE TO RELATED APPLICATIONS

This nonprovisional application is a continuation of and claims priority to PCT International Application No. PCT/US2023/025422, entitled “ROBOTIC AND AI-AUGMENTED IN SITU DETECTION AND DISINFECTION PLATFORM,” filed on Jun. 15, 2023, by the same inventors, which claims priority to provisional application No. 63/366,445, entitled “Robotic and AI-Augmented in situ Detection and Disinfection (RAID2) Platform,” filed on Jun. 15, 2022, by the same inventors, the entirety of which are incorporated herein by reference.

### FEDERALLY SPONSORED RESEARCH OR DEVELOPMENT

This invention was made with Government support under grant number 2030033 awarded by the National Science Foundation. The government has certain rights in the invention.

### BACKGROUND OF THE INVENTION

### 1. Field of the Invention

This invention relates, generally, to a disinfection system and platform. More specifically, it relates to a non-contact, robotic, in situ disinfection platform, such as for use on surfaces of locations (such as hospitals or clinics) to efficiently mitigate the spread of infectious diseases and other pathogens.

### 2. Technology Background

Many infectious diseases, such as airborne respiratory diseases (including SARS-CoV-2) spread through contaminated air and contaminated surfaced. For example, SARS-COV-2 particles have been shown to persist on a variety of surfaces for several days, including non-porous plastics, metals, ceramics, textiles, and nitrile gloves; airborne particles can also persist for hours. Approximately three years into the COVID-19 pandemic, at least 670 million confirmed cases had been recorded worldwide, with more than 6.8 million deaths associated with COVID-19.

As a result, and throughout the pandemic, people increasingly turned to household disinfectants and sanitizers to prevent the spread of airborne particles via sterilization. Specifically, the COVID-19 pandemic led to a rise in the consumption of quaternary ammonium compounds (QACs), hydrogen peroxide, bleach (sodium hypochlorite), alcohols, acids, and phenolic compounds. In addition, during 2020, sales of hand sanitizers and wipes grew by over 5,600% compared to the previous year.

While such sterilization compounds typically protect users from infection, significant increases in disinfectant exposure are associated with increased health and environment concerns. For example, the inappropriate use of disinfectants can cause indoor air pollution, dermatitis, allergic rhinitis, respiratory problems, and increases in asthma. According to the Centers for Disease Control and Prevention, from January 2020 to March 2020, poison control centers received over 45,000 exposure calls related to the misuse of cleaners and disinfectants, representing an overall increase of over 20% from the previous year. Inappropriately mixing chemicals or ingesting chemicals can cause poisoning and can result in death. In addition, the use of massive quantities of disinfectants poses a threat to the environment, bodies of water, soil, wildlife, and biodiversity.

Moreover, during outbreaks associated with airborne or surface pathogens, large quantities of personal protective equipment (PPE) are typically consumed. These types of PPE include masks, face shields, coveralls, and gloves, and are typically used for only a short period of time before disposal, which can result in an environmental burden. For example, since the start of the COVID-19 pandemic, more than 8.4 million tons of pandemic-associated plastic waste has been generated, with more than 25,000 tons of such waste entering the ocean. In addition, due to the lack of efficient, affordable, and user-friendly PPE sterilization solutions, PPE is used between patients and without proper sterilization, risking cross-infection.

Some attempts have been made to sterilize surfaces and PPE to prevent or mitigate the spread of infection. For example, chemical disinfectants, heating, ultraviolet (UV-C) radiation, and x-ray radiation are commonly used in sterilization methods. However, these techniques fail to adequately treat surfaces, air, and PPE in a way that is environmentally friendly, user-friendly, effectively, affordable, and not harmful to the user. For example, heating techniques consume a large amount of energy, damage heat-sensitive surfaces, and can only treat limited spaces. In addition, UV-C and x-ray radiation techniques involve UV-C rays and x-rays, which are carcinogenic and require additional protection components for users, thereby increasing costs and lowering efficiency. Moreover, while robotics employing UV-C radiation have been deployed in hospital settings, the devices are complex and bulky (such as having heights of over 1.5 meters), thereby similarly increasing costs and limiting applications to specific large spaces.

In addition, the sanitization of indoor air is essential to suppress the spread of airborne diseases. Traditional methods of indoor air sanitization involve the use of hydrogen peroxide sprays or UV-C lamps for a long period of time (such as 60 minutes), which are designed to prevent cross-infection in hospital and clinical settings. However, such methods are time-consuming and inefficient, requiring the evacuation of a space during the sanitization period. For spaces that cannot be regularly evacuated for sanitization (such as residential homes and other indoor facilities), traditional sanitization systems are limited to natural ventilation and to filtration through a heating, ventilation, and air conditioning (HVAC) system. However, the effectiveness of such systems is limited, and HVAC systems can further spread pathogens throughout the space, particularly when filtration is ineffective or less effective than desired.

Accordingly, what is needed is non-contact, robotic, in situ disinfection platform, such as for use on surfaces and equipment, to efficiently mitigate the spread of infectious diseases and other pathogens. However, in view of the art considered as a whole at the time the present invention was made, it was not obvious to those of ordinary skill in the field of this invention as to how the shortcomings of the prior art could be overcome.

While certain aspects of conventional technologies have been discussed to facilitate disclosure of the invention, Applicant in no way disclaims these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF SUMMARY OF THE INVENTION

The long-standing but heretofore unfulfilled need for a non-contact, robotic, in situ disinfection platform, such as for use on surfaces and equipment is now met by a new, useful, and nonobvious invention.

The present invention includes an autonomous disinfection platform including a front surface opposite a rear surface with a body extending between the front surface and the rear surface. A pair of wheels are secured to the body, with the pair of wheels being configured to translate the platform along a ground surface. The platform includes an arm extending in a direction away from one of the front surface and the rear surface. In an embodiment, the arm is pivotably attached to the body of the platform. The arm terminates at a discharge, with the discharge being configured to be disposed at a height above the ground surface. A power source is electrically coupled to the discharge, with the power source being configured to transmit a discharge voltage and a discharge current to the discharge. The autonomous disinfection platform is configured to treat, via the discharge voltage and the discharge current emitted from the discharge, a pathogen within an environment surrounding the platform.

In an embodiment, the arm is a disinfection arm that is secured to the rear surface, and the platform includes an opposing detection arm secured to the front surface, such that the detection arm extends in a direction away from the front surface and away from the disinfection arm. In an embodiment, a pair of grasping components is disposed at a terminal end of the detection arm. An embodiment of the platform includes an onboard detection chamber disposed on the platform proximate to the front surface thereof, with the onboard detection chamber being configured to receive a sample from the detection arm.

In an embodiment of the platform, the pair of wheels is a first pair of wheels secured to the body proximate to the front surface, and the platform includes a second pair of wheels secured to the body proximate to the rear surface. In an embodiment, the platform includes a wheel gear mechanically coupled to each wheel, and a motor gear mechanically coupled to each wheel gear, with each wheel gear having a diameter greater than a diameter of each motor gear.

In an embodiment, the discharge voltage is approximately 25 kV. An embodiment of the discharge current is approximately 0.05 mA. In an embodiment, the discharge is made of tungsten. An embodiment of the discharge includes a terminal ejection aperture, the terminal ejection aperture having a diameter of approximately 100 mm.

In an embodiment, the platform includes a range sensor secured to an outer surface of the body of the platform, such that the range sensor measures a distance between the platform and a surrounding surface. An embodiment of the platform includes an onboard computing device in electronic communication with the range sensor, the onboard computing device having a processor. In an embodiment, the onboard computing device includes a non-transitory computer-readable medium operably coupled to the processor, the computer-readable medium having computer-readable instructions stored thereon. When executed by the processor, the instructions include receiving, at the processor and from the range sensor, an initial scan data of the environment surrounding the platform; calculating, via the processor and based on the initial scan data, a target path for the platform within the environment surrounding the platform; receiving, at the processor and from the range sensor, an updated scan data of the environment surrounding the platform; and based on a determination that an external object intersects with the target path for the platform, calculating, via the processor, a diversion from the target path for the platform.

The present invention includes an autonomous disinfection system including an autonomous disinfection platform. The autonomous disinfection platform includes a front surface opposite a rear surface with a body extending between the front surface and the rear surface. A pair of wheels is secured to the body, the pair of wheels being configured to translate the platform along a ground surface. A range sensor is secured to an outer surface of the body of the platform, such that the range sensor measures a distance between the platform and a surrounding surface. The platform includes an arm extending in a direction away from one of the front surface and the rear surface, with the arm terminating at a discharge. The discharge is configured to be disposed at a height above the ground surface. A power source is electrically coupled to the discharge, with the power source being configured to transmit a discharge voltage and a discharge current to the discharge.

The autonomous disinfection system includes an onboard computing device in electronic communication with the range sensor. The onboard computing device includes a processor and a non-transitory computer-readable medium operably coupled to the processor. The computer-readable medium includes computer-readable instructions stored thereon that, when executed by the processor, cause the onboard computing device to execute instructions. The instructions include receiving, at the processor and from the range sensor, an initial scan data of the environment surrounding the platform; calculating, via the processor and based on the initial scan data, a target path for the platform within the environment surrounding the platform; receiving, at the processor and from the range sensor, an updated scan data of the environment surrounding the platform; based on a determination that an external object intersects with the target path for the platform, calculating, via the processor, a diversion from the target path for the platform.

An object of the invention is to safely and effectively sanitize surfaces, equipment, and air via an unmanned, autonomous, and automated platform, thereby minimizing or eliminating pathogen spread while simultaneously minimizing exposure to sanitization products. Other objects of the invention include reducing the use of disinfectants, medical waste production, and the spread of infectious diseases within locations such as hospitals and clinics.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1 is an orthogonal view of an autonomous sterilization platform, in accordance with an embodiment of the present invention.

FIG. 2 schematically depicts: a corona discharge treatment setup (in section A); a corona discharge air sterilization setup (in section B); and corona discharge sterilization experiment (in section C).

FIG. 3 graphically depicts log reductions of corona discharge on different surfaces when experimentally contaminated with E. coli (in the top graph) and with spores (in the bottom graph), including images of lysogeny broth plates before and after treatment, in accordance with an embodiment of the present invention.

FIG. 4 graphically depicts: corona discharge sterilization effectiveness of E. coli on N95 masks at different atmospheres (in section A); spectra generated by corona discharge under N₂ flow (in section B); and spectra generated by corona discharge by corona discharge under O₂ flow (in section C).

FIG. 5 graphically depicts: heat impacts of corona discharge on a target surface prior to use (in section A); heat impacts of corona discharge on the target surface after 3 minutes of use (in section B); heat impacts of corona discharge on the target surface after 7 minutes of use (in section C); ultraviolet emission spectroscopy values resulting from corona discharge when the horizontal detection distance ranges from 5.5 cm to 9.5 cm (in section D); and ozone density values resulting from corona discharge after 2 minutes, 4 minutes, 6 minutes, and 8 minutes (in section E).

FIG. 6 graphically depicts temperature changes resulting from corona discharge (in sections A, B, and C).

FIG. 7 depicts an experimental path of an autonomous sterilization platform within an obstacle course in a given space.

### DETAILED DESCRIPTION OF THE PREFERRED EMBODIMENT

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

As used in this specification and the appended claims, the singular forms “a,” “an,” and “the” include plural referents unless the content clearly dictates otherwise. As used in this specification and the appended claims, the term “or” is generally employed in its sense including “and/or” unless the context clearly dictates otherwise. All numerical designations, including ranges, are approximations which are varied up or down by increments of 1.0 or 0.1, as appropriate. It is to be understood, even if it is not always explicitly stated that all numerical designations are preceded by the term “about.” As used herein, “about,” “approximately,” or “substantially” refer to being within an acceptable error range for the particular value as determined by one of ordinary skill in the art, which will depend in part on how the value is measured or determined. As used herein, the terms “about,” “approximately,” and “substantially” refer to ±10% of the numerical; it should be understood that a numerical including an associated range with a lower boundary of greater than zero must be a non-zero numerical, and the terms “about,” “approximately,” and “substantially” should be understood to include only non-zero values in such scenarios.

The phrases “in some embodiments,” “according to some embodiments,” “in the embodiments shown,” “in other embodiments,” and the like generally mean the particular feature, structure, or characteristic following the phrase is included in at least one implementation. In addition, such phrases do not necessarily refer to the same embodiments or different embodiments.

The present invention includes an autonomous pathogen detection and disinfection mobile platform having a small footprint, such that embodiments of the platform are deployable within interior spaces such as hospital, clinics, and other healthcare facilities. The platform includes a detection chamber that efficiently identifies a presence of a pathogen, such as a microorganism. The platform also includes a disinfection discharge component, such as a discharge needle, that treats a surface that includes a presence of a pathogen. As an autonomous mobile device, the platform includes sensors, cameras, or similar devices to detect surrounding surfaces, such that a computing device disposed on the platform plots a dynamic path for the platform based on received feedback. The platform will be described in greater detail in the sections herein below.

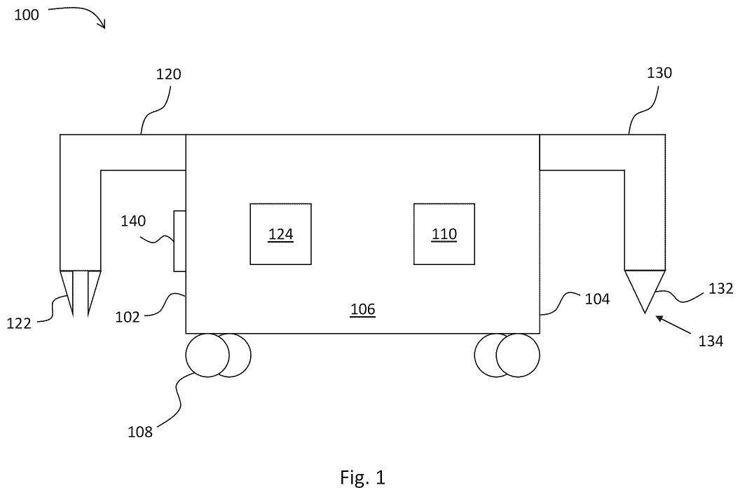

As shown in FIG. 1, an embodiment of platform 100 includes front surface 102, rear surface 104, and a pair of opposing side surfaces 106; in some embodiments, platform 100 has a total volume of less than one cubic foot. Moreover, embodiments of platform 100 are entirely self-contained, with platform 100 including one or more onboard computing devices 110 (which can include executable machine learning programs to improve platform 100 traversal and pathogen detection and treatment), detection components, disinfection components, sensing components, and other tools that provide for platform 100 to be autonomous and automated without the need for human intervention or users. These components will be described in the sections below.

Embodiments of platform 100 include two sets of wheels 108 on each side thereof, including a front wheel set and a rear wheel set. In such embodiments, each wheel 108 is connected to a wheel gear having an associated wheel gear diameter, with each wheel gear being connected to a motor gear having an associated motor gear diameter; each motor gear is connected to a motor. Each of the wheel gear diameters is greater than each of the motor gear diameters. For example, in an embodiment, each wheel gear is approximately equal in diameter, and each motor gear is approximately equal in diameter, with each wheel gear having a diameter of 3.5 inches, and with each motor gear having a diameter of 0.5 inches. As such, in an embodiment, the motor runs at full power while maintaining a slow speed of approximately 0.2 ft/s; slower speeds enhance a detection and a disinfection efficacy of platform 100, which will be described in greater detail below. In addition, in an embodiment, the motor is electrically coupled to a power source, such as a battery (for example, in an embodiment, a 12-volt battery), and to a high-voltage transformer. The power source and the high-voltage transformer are secured to the robot and are in electrical communication with the discharge needle to create a disinfecting discharge (such as a corona discharge, or CD) through the discharge needle, as will be described in greater detail in the sections below.

It should be appreciated that other transportation components are contemplated herein that function similarly to wheels disposed along a ground surface; for example, embodiments of platform 100 can include one or more turbines to hover at least temporarily above a ground surface; continuous tracks to traverse along a ground surface; independent legs to traverse along a ground surface; hulls or other floating surfaces to traverse along a fluid-based surface; and combinations thereof.

Platform 100 includes at least one arm 130 attached to a surface of platform 100, such that arm extends in a direction away from platform 100; for example, in an embodiment, arm 130 is attached to rear surface 104 of platform 100. Arm 130 includes a terminal end, which is configured to be disposed at a height above a ground surface. In an embodiment, discharge needle 132 is attached to arm 130. In addition, in an embodiment, arm 130 includes at least two grasping components 122 (such as claws) at terminal end thereof, with discharge needle 132 being held between the at least two grasping components 122. Discharge needle 132 will be described in greater detail in the sections below. In an embodiment, arm 130 is a static arm; however, it should be appreciated that dynamic and mobile arms (such as arms capable of movement in one direction, two directions, a plurality of directions, and across 360° degrees) are also contemplated herein.

In an embodiment, platform 100 includes a plurality of arms, such as including a detection arm 120 attached to a surface of platform 100 and disinfection arm 130 attached to a surface of platform 100. For example, in an embodiment, platform 100 includes detection arm 120 attached to front surface 102 of platform 100 and disinfection arm 130 attached to rear surface 104 of platform 100. In such an embodiment, as platform 100 traverses within a given space (such as along a ground surface of an interior environment) in a forward direction, pathogens can be detected via detection arm 120 of platform 100, with disinfection arm 130 being subsequently utilized to treat the pathogens, such as via elimination or minimization of pathogens and/or pathogen spread.

In an embodiment, at least one range sensor 140 is secured to platform 100 to measure a distance between a surface of platform 100 and a surface of a surrounding environment (such as a wall, object, or other obstacle), with the measured distance being transmitted from the at least one range sensor 140 to a computing device disposed on platform 100 to calculate and guide a path of platform 100. For example, in an embodiment, sensor 140 is secured to front surface 102 of platform 100 to measure distances between front surface 102 of platform 100 and a surrounding environmental surface. In some embodiments, a plurality of sensors are secured to platform 100; for example, in an embodiment, platform 100 includes a front sensor 140 secured to front surface 102 thereof, a first side sensor 140 secured to first side 106 thereof (such as a left side), and a second side sensor 140 secured to second side 106 thereof (such as a right side). As such, embodiments of platform 100 include sensors 140 to measure distances between platform 100 and any surrounding surface, such that a path of platform 100 (such as along a ground surface) can be planned by computing device 110 to avoid contact between platform 100 and the surrounding surface.

Specifically regarding the path-guiding of platform 100, computing device 110 first generates a targeted designated path for platform 100 within a given space, such as an interior environment. As such, during an initial step, computing device 110 receives (or otherwise contains a stored copy of) a map of the given space; for example, in an embodiment of platform 100, computing device 110 receives an initial scan data including a map of surfaces disposed within the given space from at least one range sensor 140, as described in detail above. Next, computing device 110 calculates a path for platform 100 within the given space based on the initial scan data, and computing device 110 transmits an instruction to the motor to translate platform 100 along the calculated path. The path is calculated such that, based on measured distances between platform 100 and surfaces within the given space, contact is avoided between platform 100 and the detected surfaces. If computing device 110 calculates a diversion from the path (either based on the initial scan data or based on updated scan data received by computing device 110 during the traversal of platform 100 within the given space), computing device 110 transmits an instruction to the motor to divert from a planned path to avoid contact with a surrounding surface and to return to the planned path after avoiding such contact.

As such, in embodiments of platform 100, computing device 110 continuously autonomously guides platform 100 within the given space to avoid contact with any obstacles or other surfaces that intrude upon a planned path. In embodiments, computing device 110 transmits the initial scan data, map data, calculated path(s), and/or diversion(s) to a central data collection station that is separate from platform 100. In such embodiments, the central data collection station (which is in wireless communication with a plurality of platforms 100) receives the data from a first platform 100 and transmits the data to one or more of the plurality of platforms 100. As such, in embodiments, a plurality of platforms 100 (such as a swarm) can be deployed and implemented within the same given space. In embodiments, the central data collection station, after receiving map data relating to the given space, divides the given space into a plurality of regions, with each of the plurality of platforms 100 being confined to one of the plurality of regions. The plurality of regions are calculated such that the plurality of regions do not overlap, and such that contact is avoided between different platforms 100 of the plurality of platforms 100.

As noted in the sections above, embodiments of platform 100 are configured to detect and/or disinfect a target surface. Target surfaces include conductive surfaces and non-conductive surfaces, including personal protective equipment (PPE) materials (such as cloth masks, face shields made of polyethylene terephthalate (PET), coveralls made of polypropylene (PP), alumina (and similar ceramic surfaces), and stainless steel (and similar conductive surfaces)).

For example, in an embodiment, platform 100 includes detection arm 120 attached to a surface thereof. In an embodiment, a moist swab (or a similar collection device) is secured to detection arm 120, such as between two grasping components 122; based on an instruction from computing device 110, detection arm 120 contacts the moist swab with a target surface to collect a sample from the target surface. The swab is then inserted into testing module 124 of platform 100 to detect the presence of a targeted microbe; in an embodiment, such detection occurs in approximately 2-5 minutes using a multiplexed nucleic acid-based biosensor (MNB). For example, in an embodiment, testing module 124 includes a SARS-COV-2 antigen detector using virus DNA-sequences as capture (DCS) for SARS-COV-2 S and N antigens. In an embodiment, a 3-step gravitational flow-through protocol using a DCS-matrix and a gDCS probe is used for testing within testing module 124. It should be appreciated that in other embodiments, non-contact detection devices can be used in conjunction with detection arm 120. The results of testing are transmitted from testing module 124 to computing device 110; based on the results, in some embodiments, computing device 110 issues an instruction to disinfection arm 130 of platform 100 (as will be described below).

As noted above, embodiments of platform 100 are configured to treat a target surface to eliminate or minimize pathogen presence, growth, or spread associated with the target surface. As such, in an embodiment (and as shown in particular in FIG. 1), platform 100 includes disinfection arm 130 that extends away from a body of platform 100. In some embodiments, disinfection arm 130 includes or otherwise terminates with discharge 132, such as a discharge needle or other similar discharge device. Discharge 132 is configured to reside at a height above the target surface (such as a ground surface); for example, in some embodiments, discharge 132 is configured to reside at a height of approximately 3.5 cm above the target surface. However, it should be appreciated that in other embodiments, discharge 132 may be closer to or further from the target surface.

In embodiments, discharge 132 includes a diameter of approximately 100 mm; however, it should be appreciated that the diameter of discharge 132 can range from approximately 50 mm to approximately 150 mm. Moreover, in some embodiments, the diameter of discharge 132 is dynamic and changes based on the dimensions of the target area, such as by altering the diameter of discharge tip 134.

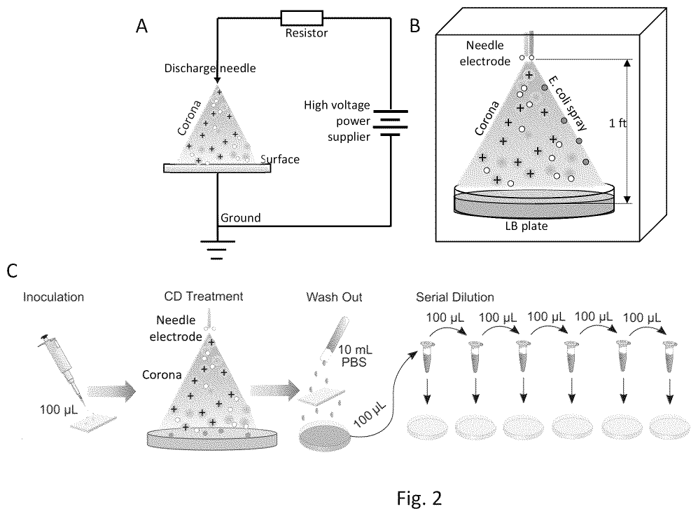

Discharge 132 is electrically coupled to the power supply of platform 100 (or a similar power supply in communication with platform 100), such that discharge 132 receives a voltage and a current from the power supply (such as a high voltage direct current power supply; an embodiment schematic of discharge 132 is shown in FIG. 2). In some embodiments, discharge 132 is made at least in part of an electrically conductive material, such as tungsten or a similar conductive material. As such, in some embodiments, discharge 132 is an electrode that is electrically coupled to the power supply, such that discharge 132 is configured to emit a discharge voltage and a discharge current toward the target surface. For example, in an embodiment, discharge 132 is configured to emit a discharge voltage of approximately 25 kV and a discharge current of approximately 0.05 mA, having an output power of approximately as low as 1.25 W. However, it should be appreciated that in some embodiments, discharge 132 is configured to emit a discharge voltage of up to approximately 60 kV and a discharge current of up to approximately 0.20 mA.

As such, via discharge 132, platform 100 disinfects and sterilizes the target surface via the emission from discharge 132. Specifically, discharge 132 emits corona discharge (CD) that ruptures cell membranes and results in DNA damage of treated pathogens on the target surface. As tested by a spectrometer and an ozone detector, reactive nitrogen series (RNS), reactive oxygen series (ROS), and ozone are generated from discharge to treat the target surface, with chemical reactions including the following:

e+O₂→e+O(³P)+O(³P)

O+O₂+M→O₃+M

N+O+N₂→NO+N₂

NO+O₃→NO₂+O₂

NO₂+O₂+hv→O₃+NO

H₂O+O₃→O₂+2OH

The OH radical weakens the barrier function of the unsaturated fatty acid lipid bilayer on the cell membrane, allowing ions and polar compounds to enter the cell. The proteins embedded in the lipid bilayers that can control the passage of various compounds are also susceptible to oxidation when exposed to the ROS and radical-rich environment in the corona, leading to additional damage to the cell membrane. With the integrity of the cell membrane damaged and paths opened, the reactive species generated by corona can directly interact with subcellular biomaterials in the microorganisms, leading to their destruction and inactivation, and thereby resulting in treatment of the target surface.

### Experimental Methods

As shown in FIG. 2, platform 100 (as described in greater detail above) was tested on a variety of target surfaces, including cloth masks, face shields made of PET, coveralls made of PP, alumina, and stainless steel. The specimens of each tested surface were cut into squares measuring 5 cm by 5 cm and mounted on a ground electrode made from a stainless steel plate. Discharge 132 was set vertically above the center of the specimen at 3.5 cm.

E. coli was chosen as the major microorganism in most sterilization tests to increase experiment efficiency due to the relatively short culturing time and low biosafety concern. The E. coli solution was spread evenly to form a uniform layer covering the entire surface area of the specimens. Via discharge 132, CD was applied to the samples for 7.5 minutes at a discharge voltage of 25 kV and discharge current of 0.05 mA. Some experiments included CD treatment for more than one cycle. Five minutes of resting time was added between each cycle. After CD treatment, the surviving colonies were washed from the sample with phosphate-buffered saline (PBS). 100 μL of the resulting solution was then added to 900 μL of PBS; this process was repeated to obtain six serial dilutions. 100 μL of each dilution was smeared onto a lysogeny broth (LB) agar plate for incubation at 37° C. After 20 hours, the surviving colony-forming units (CFU) were counted. All experiments were conducted in triplicate.

Sterilization experiments were also performed on spores; specifically, experiments were performed on Geobacillus stearothermophilus, which produces heat-resistant spores and possesses thicker cell walls than E. coli (resulting in better tolerance against membrane rupture and DNA leakage). G. stearothermophilus cells were prepared using 100 mL of nutrient broth (to cultivate non-fastidious microorganisms; including 3 grams of beef extract and 5 grams of peptone per liter) and incubated overnight at 50° C. at 120 revolutions per minute (rpm), reaching an initial concentration of 104 CFU/mL. The same treatment against E. coli was applied to evaluate the sterilization of CD against G. stearothermophilus.

CD generated in different atmospheres were compared to evaluate the necessity of gas input to enhance the sterilization effect. During testing, the atmosphere of the CD process was controlled by initiating sterilization when a controllable gas flow was turned on. The gas nozzle was set in parallel with discharge 132. The flow pressure was set at 20 pounds per square inch (psi). Both N₂ and O₂ gases were tested. In addition, to evaluate the impact of humidity, mist was injected from a humidifier into the test chamber, with humidity being monitored via a humidity sensor. To evaluate the capability of CD for air sterilization, an E. coli solution with a concentration of 103 CFU was sprayed along the direction of CD; the distance between the spray nozzle and the LB plate was set at 30 cm. The spray zone was enclosed in a transparent chamber to avoid leakage or contamination of the surroundings.

The optical emission spectrometry (OES) spectrum of CD was acquired using a spectrometer (with a fiber optic cable positioned at horizontal distances of 5.5 cm, 7.5 cm, and 9.5 cm from discharge). The ion density from discharge 132 was measured using an air ion counter. The measurements were conducted using positive polarity, and the device was positioned at a parallel distance of 7.5 cm from discharge tip 134. Ozone generation was measured using an ozone meter at 0 cm to 5 cm away from the center of the corona (measured horizontally) before the corona was turned on, with additional measurements taking place after the corona was turned on, at 2 minutes, 4 minutes, 6 minutes, and 8 minutes.

### Experimental Results

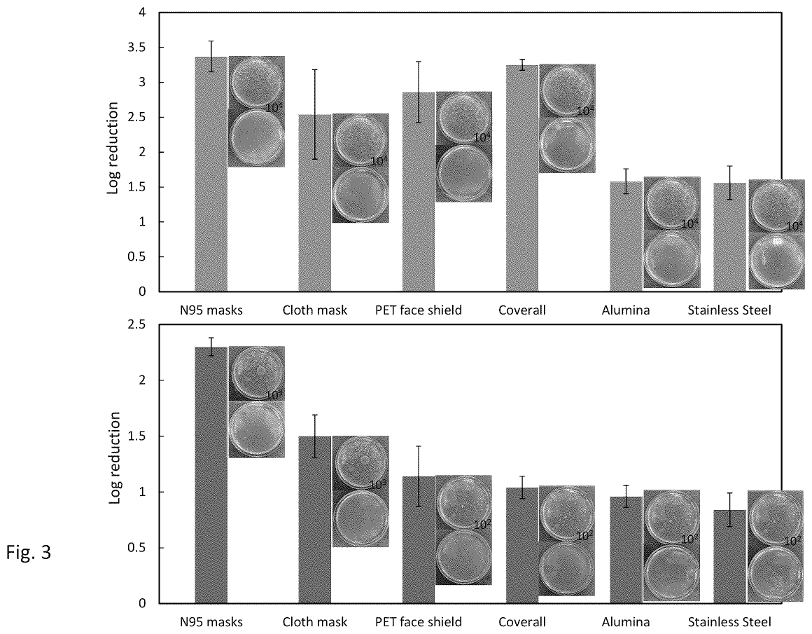

As noted above, platform 100 was tested on a variety of target surfaces, including cloth masks, face shields made of PET, coveralls made of PP, alumina, and stainless steel. Log reductions of CD on each target surface are shown in FIG. 3, with log reductions for E. coli being shown in the top graph and log reductions for spores being shown in the bottom graph.

### E. Coli Testing

Experiments showed that after 2 cycles of CD treatment, the average log reduction for E. coli on N95 masks reaches as high as 3.37. The log reduction for cloth masks turned out to be lower than for N95 masks, with an average value of 2.54, which is caused by the hydrophilic properties and the thinner thickness of the cotton cloth. The E. coli solution-soaked part of the cloth material, which gathered at the surface of the substrate underneath, led to less exposure of E. coli to CD. However, an average log reduction of 2.54 is better than regular washing, the most common cleaning method for cloth masks. Additionally, compared with washing, CD treatment is a non-contact and dry process, skipping the long-time drying procedure and reducing the waiting time from hours to minutes between each use.

Face shields and coveralls are another two important PPEs for protecting healthcare workers from infectious diseases. Face shields are normally reused, which requires a sterilization process, such as alcohol spraying or UV-C radiation, which may cause material degradation, washing, which consumes a large amount of water and detergent, or hydrogen peroxide vapor (HPV), with high temperatures may cause damage to PET material. It was confirmed that CD treatment can achieve an average log reduction of 2.86 as a contactless, heatless solution without impacting the transmittance of the face shield. In addition, a log reduction of 3.25 on coveralls was provided by CD; the large area scanning capability of platform 100 provides for the disinfection of large-size PPE and biowaste at the industrial level, thereby alleviating concerns for non-degradable plastic pollution and biowaste-caused infections related to the use of coveralls.

In addition to PPE made by polymers, sterilization experiments were also conducted on ceramics and metals by using alumina and stainless steel as representatives. However, the contact angle between the E. coli solution and the smooth alumina and stainless-steel surfaces was large. Accordingly, it was difficult to minimize the droplet size, resulting in droplets with diameters larger than 1 mm and limiting the exposure of E. coli to CD. The test average log reductions for millimeter droplets on alumina and stainless steel were 1.58 and 1.56, respectively, which still provided for the sterilization of daily-used surfaces.

### Spore Testing

In addition, as noted above, CD was tested on spores of G. stearothermophilus, a type of gram-positive bacterium that is widely distributed in soil, hot springs, and ocean sediment, and that is the cause of the flat, sour spoilage of canned liquid foods. The G. stearothermophilus spore is one of the most disinfection-resistant spores of aerobic microorganisms, highly resistant to many physical treatments and chemical agents, including heat, drying, radiation, and chemicals. They are often used as biological indicators to evaluate the effectiveness of sterilization processes.

The log reductions of CD against spores on different surfaces were evaluated, as shown in the bottom graph of FIG. 3. It was confirmed that the log reduction for G. stearothermophilus on N95 masks can reach as high as 2.3 on average. The log reduction on cloth masks is around 1.5, impacted by the soaking of large droplets. Face shields and coveralls were sterilized at a log reduction of 1.14 and 1.04, respectively. The log reductions on alumina and stainless-steel surfaces are also approximately 1. Since the surfaces of the face shields, coveralls, alumina, and stainless steel are all much smoother compared with the porous masks, the droplets of the microorganism solutions are normally larger than 1 mm in diameter because of surface tension. It is expected that the log reduction in the real application, when the droplet size is in the micrometer range, can be higher, since the microorganisms can be better exposed to CD. The sterilization effect of CD was also tested on Pichia pastoris strain SMD1163, which is another challenging microorganism for sterilization. A log reduction of approximately 1.5 on most surfaces was reached.

The results indicated that CD is an effective broad-spectrum sterilization solution for the majority of surfaces. More attractively, the regular sterilization practice for G. stearothermophilus requires a 15-minute autoclave treatment at 121° C., which is a time- and energy-consuming process that risks damaging the material because of the long-time heating. In comparison, CD is a contactless, non-chemical, and low-energy consumption method (down to 1.25 W), which makes it very promising for addressing sterilization challenges in soil, food, and other surfaces.

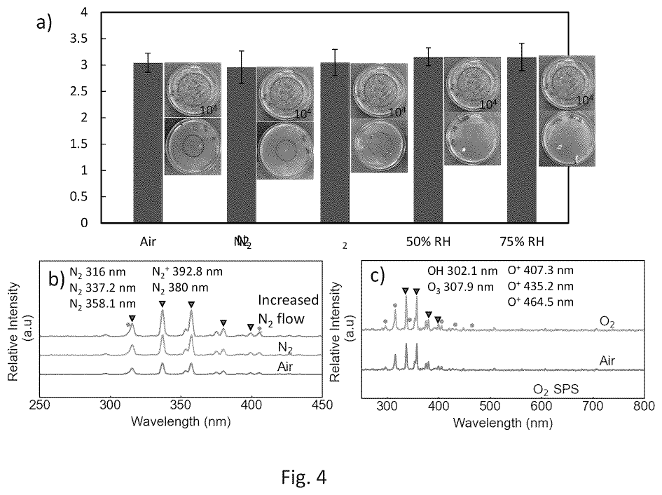

Since platform 100 operates in an ambient environment, the surrounding atmosphere may significantly impact its sterilization effectiveness. It is meaningful to evaluate whether injecting N₂ or O₂ gas can help increase sterilization effectiveness. As shown in FIG. 4A, N₂ and O₂ gas injected corona has comparable sterilization efficacy to air corona. The spectra data depicted in FIG. 4B and 4C showed higher intensities of reactive nitrogen species (RNS) and reactive oxygen species (ROS) species, respectively.

Humidity is another aspect that may impact the strength and sterilization effectiveness of corona. In FIG. 4A, the log reductions were compared at 50% relative humidity (RH) and 75% RH, with results showing that the log reduction difference is minor. Therefore, the sterilization effectiveness of corona is relatively resilient against changes in gas content or humidity level. Also, the corona generated in ambient air is more cost-effective, portable, and convenient to use, making it the best choice for daily surface sterilization applications.

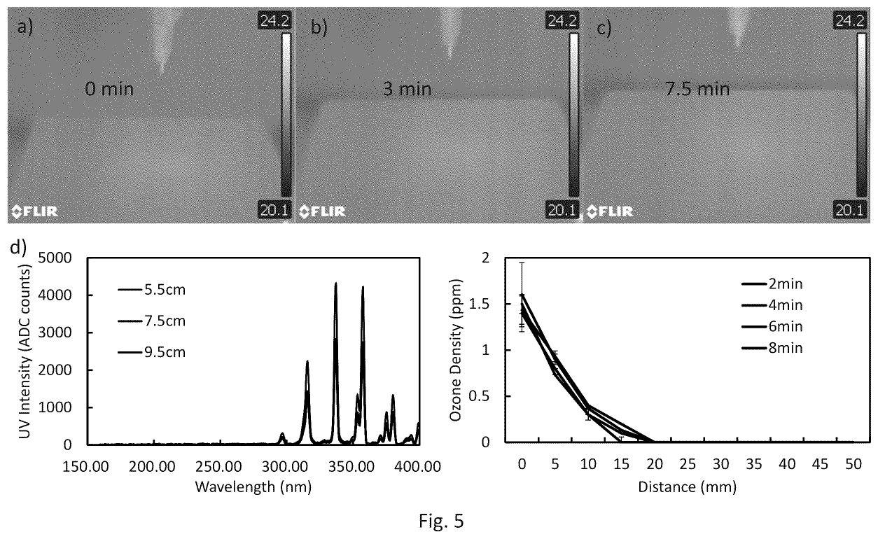

To evaluate the air sterilization effectiveness of CD, the E. coli solution was sprayed onto the LB plate from a distance of 30 cm. It was confirmed that when spraying E. coli solution in parallel with CD, the contaminated air can be disinfected with a log reduction of approximately 0.9 (FIGS. 5A and 5B). As it is difficult to experimentally evaluate the effective range of corona sterilization, a simulation was utilized to address this problem. As elaborated in FIG. 5C, the corona was generated from discharge tip. The size of the corona increases rapidly with time until it reaches the target surface, such as a ground substrate. The bell-shaped corona was fully established in a time as short as 10 ms, which means when needed, corona is an “instant” sterilization solution once turned on.

Moreover, when the distance between discharge tip 134 and the target surface is 3.5 cm, the covered area can have a diameter of 7 cm. With the increase in discharge-surface distance, the entire size of the 3D bell-shaped corona will increase, and the surface area covered by it will increase accordingly. Also, the CD can be generated by multiple discharges 132, thin wires, and 3D-shaped electrodes. Therefore, corona can be used as an “instant” sterilization solution for a large volume of air and large surface areas. CD can potentially overcome the problems of conventional air filters used in ventilating systems, which cannot prevent direct infection between people or require a high cost for filter replacement.

### Safety Results

The heat, UV-C, and ozone emitted by corona are the three major health concerns when considering daily applications of platform 100 for surface and air sterilization, especially when other occupants or users are present. Each of heat, UV-C, and ozone emissions were measured while the corona was turned on for different durations of time.

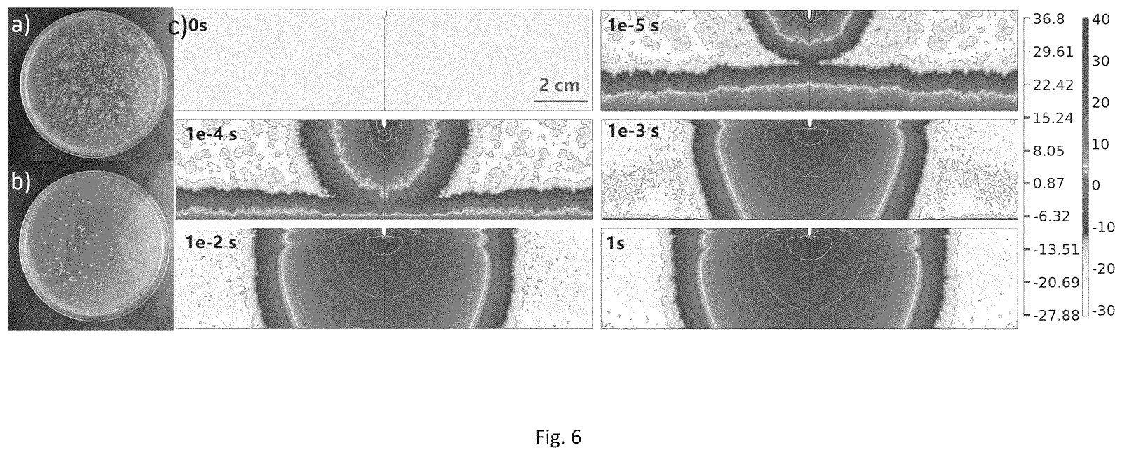

First, an infrared camera was utilized to evaluate the temperature change around the corona, especially on the target surface. As such in FIGS. 6A-6C, the temperature changes before and after 7.5 minutes of corona treatment were very minor, indicating the heat impact of corona on the treated surfaces is negligible.

Second, the carcinogenic UV-C represents a wavelength in the range of approximately 100 nm to 290 nm. The spectrum data shown in FIG. 5D shows that the UV-C emission from the corona is also negligible. In addition, the waves in the range of 290 nm to 400 nm decrease with the increase in distance between the corona and the target surface. Therefore, the UV-C radiation hazards are low enough to allow close proximity between an occupant and platform 100 using corona without extra protection being required.

Finally, the ozone emission around the corona at different distances was measured and plotted as shown in FIG. 5E. The ozone concentration reduces dramatically from approximately 1.5 ppm to 0 when the distance away from the center of the corona (the horizontal distance from the discharge needle) increased from 0 to 20 mm. As simulated in FIG. 5C, the ozone emission is still within the range of corona. Therefore, the ozone emission from CD is also shown to be acceptable for daily use while other occupants are present.

Overall, the thermal, UV-C, and ozone impacts of platform 100 using corona are all within the safe range for daily use, overcoming the limitations of UV-C, ozone, and chemical treatments, as well as dry and wet heating. Compared with most other sterilization solutions, including heat, UVC, and chemical disinfectants, CD is an efficient, non-contact, low-temperature, non-toxic, and user-friendly sterilization solution that allows for daily and repeated use, leading to negligible material damage and impact on other occupants. No extra protection precautions are needed, which makes platform 100 more convenient and affordable than previous attempted solutions. Platform 100 can also increase space efficiency by allowing occupants to co-occupy a space with platform 100.

### Autonomy Results

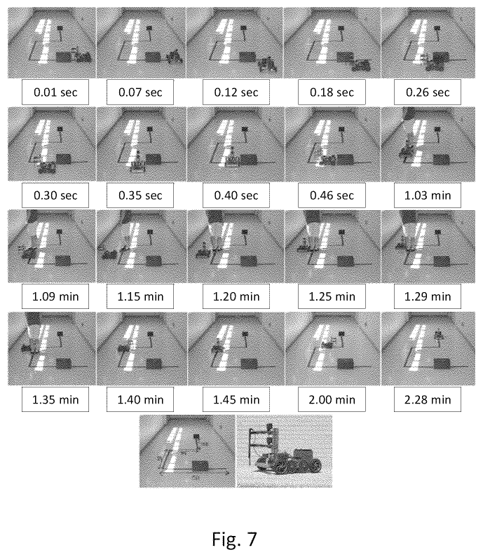

To test and validate platform 100, an obstacle course was created (FIG. 7) that includes many different turns and obstacles, specifically one static and one moving obstacle. Once platform 100 detects an obstacle with its one or more ultrasonic rangefinder sensors, platform 100 is programmed to move around the obstacle. Once platform 100 clears the obstacle, platform 100 continues along its original path. When combined with the benefits of CD mentioned above, the low-cost and user-friendly CD platform 100 has the potential to be widely used in areas such as hospitals, public areas, schools, and homes, and can work while other occupants and users are present, significantly increasing space use and work efficiency.

### COMPUTER AND SOFTWARE TECHNOLOGY

The present invention may be embodied on various platforms. The following provides an antecedent basis for the information technology that may be utilized to enable the invention.

Embodiments of the present invention may be implemented in hardware, firmware, software, or any combination thereof, including computing device 110. Embodiments of the present invention may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read-only memory (ROM); random access memory (RAM); magnetic disk storage media; optical storage media; flash memory devices; electrical, optical, acoustical, or other forms of propagated signals (e.g., carrier waves, infrared signals, digital signals, etc.), and others.

Further, firmware, software, routines, and instructions may be described herein as performing certain actions. However, it should be appreciated that such descriptions are merely for convenience and that such actions, in fact, result from computing devices, processors, controllers, or other devices executing the firmware, software, routines, instructions, etc.

The machine-readable medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any non-transitory, tangible medium that can contain or store a program for use by or in connection with an instruction execution system, apparatus, or device. Storage and services may be on-premises or remote, such as in the “cloud” through vendors operating under the brands MICROSOFT AZURE, AMAZON WEB SERVICES, RACKSPACE, and KAMATERA.

A machine-readable signal medium may include a propagated data signal with machine-readable program code embodied therein, for example, in baseband or as part of a carrier wave. Such a propagated signal may take any of a variety of forms, including, but not limited to, electromagnetic, optical, or any suitable combination thereof. A machine-readable signal medium may be any machine-readable medium that is not a computer-readable storage medium and that can communicate, propagate, or transport a program for use by or in connection with an instruction execution system, apparatus, or device. However, as indicated above, due to circuit statutory subject matter restrictions, claims to this invention as a software product are those embodied in a non-transitory software medium such as a computer hard drive, flash-RAM, optical disk, or the like.

Program code embodied on a machine-readable medium may be transmitted using any appropriate medium, including but not limited to wireless, wireline, optical fiber cable, radiofrequency, etc., or any suitable combination of the foregoing. Machine-readable program code for carrying out operations for aspects of the present invention may be written in any combination of one or more programming languages, including an object-oriented programming language such as Java, C #, C++, Visual Basic, or the like and conventional procedural programming languages, such as the “C” programming language or similar programming languages. Additional languages may include scripting languages such as PYTHON, LUA, and PERL.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by machine-readable program instructions.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

What is claimed is:

1. An autonomous disinfection platform comprising:
a front surface opposite a rear surface with a body extending between the front surface and the rear surface;
a pair of wheels secured to the body, the pair of wheels configured to translate the platform along a ground surface;
an arm extending in a direction away from one of the front surface and the rear surface, the arm terminating at a discharge, the discharge configured to be disposed at a height above the ground surface; and
a power source electrically coupled to the discharge, the power source configured to transmit a discharge voltage and a discharge current to the discharge,
wherein the autonomous disinfection platform is configured to treat, via the discharge voltage and the discharge current emitted from the discharge, a pathogen within an environment surrounding the platform.

2. The autonomous disinfection platform of claim 1, wherein the arm is a disinfection arm that is secured to the rear surface, further comprising an opposing detection arm secured to the front surface, such that the detection arm extends in a direction away from the front surface and away from the disinfection arm.

3. The autonomous disinfection platform of claim 2, further comprising a pair of grasping components disposed at a terminal end of the detection arm.

4. The autonomous disinfection platform of claim 2, further comprising an onboard detection chamber disposed on the platform proximate to the front surface thereof, the onboard detection chamber configured to receive a sample from the detection arm.

5. The autonomous disinfection platform of claim 1, wherein the arm is pivotably attached to the body of the platform.

6. The autonomous disinfection platform of claim 1, wherein the pair of wheels is a first pair of wheels secured to the body proximate to the front surface, further comprising a second pair of wheels secured to the body proximate to the rear surface.

7. The autonomous disinfection platform of claim 6, further comprising a wheel gear mechanically coupled to each wheel, and a motor gear mechanically coupled to each wheel gear, each wheel gear having a diameter greater than a diameter of each motor gear.

8. The autonomous disinfection platform of claim 1, wherein the discharge voltage is approximately 25 kV.

9. The autonomous disinfection platform of claim 1, wherein the discharge current is approximately 0.05 mA.

10. The autonomous disinfection platform of claim 1, wherein the discharge is made of tungsten.

11. The autonomous disinfection platform of claim 1, wherein the discharge includes a terminal ejection aperture, the terminal ejection aperture having a diameter of approximately 100 mm.

12. The autonomous disinfection platform of claim 1, further comprising a range sensor secured to an outer surface of the body of the platform, such that the range sensor measures a distance between the platform and a surrounding surface.

13. The autonomous disinfection platform of claim 12, further comprising an onboard computing device in electronic communication with the range sensor, the onboard computing device having a processor.

14. The autonomous disinfection platform of claim 13, further comprising a non-transitory computer-readable medium operably coupled to the processor, the computer-readable medium having computer-readable instructions stored thereon that, when executed by the processor, cause the onboard computing device to execute instructions comprising:
receiving, at the processor and from the range sensor, an initial scan data of the environment surrounding the platform;
calculating, via the processor and based on the initial scan data, a target path for the platform within the environment surrounding the platform;
receiving, at the processor and from the range sensor, an updated scan data of the environment surrounding the platform; and
based on a determination that an external object intersects with the target path for the platform, calculating, via the processor, a diversion from the target path for the platform.

15. An autonomous disinfection system comprising:
an autonomous disinfection platform comprising:
a front surface opposite a rear surface with a body extending between the front surface and the rear surface;
a pair of wheels secured to the body, the pair of wheels configured to translate the platform along a ground surface;
a range sensor secured to an outer surface of the body of the platform, such that the range sensor measures a distance between the platform and a surrounding surface;
an arm extending in a direction away from one of the front surface and the rear surface, the arm terminating at a discharge, the discharge configured to be disposed at a height above the ground surface; and
a power source electrically coupled to the discharge, the power source configured to transmit a discharge voltage and a discharge current to the discharge; and
an onboard computing device in electronic communication with the range sensor, the onboard computing device comprising:
a processor; and
a non-transitory computer-readable medium operably coupled to the processor, the computer-readable medium having computer-readable instructions stored thereon that, when executed by the processor, cause the onboard computing device to execute instructions comprising:
receiving, at the processor and from the range sensor, an initial scan data of the environment surrounding the platform;
calculating, via the processor and based on the initial scan data, a target path for the platform within the environment surrounding the platform;
receiving, at the processor and from the range sensor, an updated scan data of the environment surrounding the platform; and
based on a determination that an external object intersects with the target path for the platform, calculating, via the processor, a diversion from the target path for the platform.
